(19) United States
(12) Patent Application Publication
(10) Pub. No.: US 2021/0171459 A1
(43) Pub. Date: Jun. 10, 2021

(54) PREPARATION OF CAPROLACTAM FROM 6-AMINO CAPROIC ACID OBTAINED IN A FERMENTATION PROCESS

(71) Applicant: Genomatica, Inc., San Diego, CA (US)

(72) Inventors: Rudolf Philippus Maria Guit, Maastricht (NL); Thomas Van Der Does, Wilnis (NL); Lourina Madeleine Raamsdonk, Nootdorp (NL)

(21) Appl. No.: 16/932,405

(22) Filed: Jul. 17, 2020

Related U.S. Application Data
(63) Continuation of application No. 14/963,114, filed on Dec. 8, 2015, now abandoned.
Application No. 14/963,114 is a continuation of application No. 13/518,647, filed on Oct. 12, 2012, now abandoned.

(30) Foreign Application Priority Data
Dec. 22, 2009 (EP) 09180383.3

(51) Int. Cl. C07D 201/08; C07D 223/10; C07D 201/16; C08G 69/16

(52) U.S. Cl. CPC C07D 201/08; C08G 69/16; C07D 201/16; C07D 223/10

(57) ABSTRACT

The invention relates to a method for preparing caprolactam comprising recovering a mixture containing 6-aminocaproic acid, from a culture medium comprising biomass, and thereafter cyclising the 6-aminocaproic acid in the presence of superheated steam, thereby forming caprolactam, wherein the weight to weight ratio carbohydrate to 6-aminocaproic acid in said mixture is 0.03 or less.

This application is a continuation of U.S. patent application Ser. No. 14/963,114, filed Dec. 8, 2015, which is a continuation of U.S. patent application Ser. No. 13/518,647, § 371(c) date of Oct. 12, 2012, which is the U.S. National Stage Application under 37 U.S.C. § 371 of International Patent Application No. PCT/NL2010/050878, filed Dec. 22, 2010, which designates the U.S. and claims the benefit of priority to EP Application No. 09180383.3, filed Dec. 22, 2009, the entire contents of which are each incorporated herein by reference.

The invention relates to a method for preparing ε-caprolactam (hereinafter caprolactam or CAP) from biochemically prepared 6-aminocaproic acid (hereinafter 6-ACA).

Caprolactam is a lactam which may be used for the production of polyamide, for instance nylon-6. Various manners of preparing caprolactam from bulk chemicals are known in the art and include the preparation of caprolactam from toluene or benzene. These compounds are generally obtained from mineral oil. In view of a growing desire to prepare materials using more sustainable technology it would be desirable to provide a method wherein caprolactam is prepared from an intermediate compound that can be obtained from a biologically renewable source or at least from an intermediate compound that is converted into caprolactam using a biochemical method. Further, it would be desirable to provide a method that has a smaller ecological footprint than conventional chemical processes making use of bulk chemicals from petrochemical origin, in particular a method that requires less energy and/or has a lower carbon dioxide emission than said conventional processes.

In WO 2005/068643 it is disclosed that caprolactam may be prepared from 6-ACA that has been prepared biochemically by converting 6-aminohex-2-enoic acid (6-AHEA) in the presence of an enzyme having α,β-enoate reductase activity. For the preparation of caprolactam from 6-ACA reference is made to U.S. Pat. No. 6,194,572.

U.S. Pat. No. 6,194,572 discloses the preparation of caprolactam by treating 6-aminocaproic acid, 6-aminocaproate ester or 6-aminocaproamide or mixtures comprising at least two of these compounds in the presence of superheated steam in which a gaseous mixture comprising caprolactam and steam is obtained, wherein the process is carried out in a cyclisation reactor in the absence of a catalyst at a temperature between 250 and 400° C. and at a pressure of between 0.5 and 2 MPa. In a preferred embodiment, caprolactam is prepared from a reaction mixture consisting of 6-aminocaproic acid, 6-aminocaproate ester, 6-aminocaproamide, optionally caprolactam and optionally oligomers of said compounds.

A method specifically directed to the preparation of caprolactam by cyclising 6-ACA obtained in a fermentative process is not described in detail in WO 2005/068643, nor is the purification of the thus obtained caprolactam.

The inventors have come to the conclusion that although it is possible to introduce the product of a biochemical process directly into the cyclisation reactor, if the direct product of a fermentative process (6-ACA in a fermentation broth) is subjected to cyclisation in the cyclisation reactor, using typical cyclisation conditions, the caprolactam yield is relatively low. Further, the inventors have come to the conclusion that it is a challenge to purify the crude caprolactam thus obtained.

It is an object of the present invention to provide a novel process for the preparation of caprolactam from 6-ACA obtained in a biochemical process, in particular such process that allows a satisfactory caprolactam yield.

Accordingly, the present invention relates to a method for preparing caprolactam comprising recovering a mixture containing 6-aminocaproic acid, from a culture medium comprising biomass, and thereafter cyclising the 6-aminocaproic acid in the presence of superheated steam, thereby forming caprolactam, wherein the weight to weight ratio carbohydrate to 6-aminocaproic acid in said mixture is 0.03 or less. In particular, said ratio may be 0.025 or less, or 0.02 or less, or 0.01 or less, or even less than 0.005. Said ratio may be 0 or more, in particular 0.001 or more. This ratio will thus be in the range of from 0 to 0.03.

The culture medium may in particular be a culture medium used for preparing 6-ACA in a fermentation process. The term ‘fermentation’ is used herein in the general sense for an industrial process wherein use is made of an organism for converting at least one (organic) substance into at least one other (organic) substance. The fermentation process can take place under aerobic, oxygen limited or anaerobic conditions.

In the fermentative process a fermentation product is obtained. This product comprises 6-ACA, biomass and typically several other components that are generally present in fermentation broths (nutrients, buffering salts, etc. and (by)products such as ethanol, glycerol, acetate etc). The inventors contemplate that it may be sufficient to separate one or more specific components from the 6-ACA, prior to cyclisation, or to carry out the fermentation under conditions that result in a low abundance of such component or components. Without being bound by theory, components that are considered to potentially affect the yield of caprolactam include: carbohydrates, in particular monosaccharides from the group of hexoses and pentoses, oligomers thereof and polymers thereof, more in particular glucose, fructose, mannose, sucrose, lactose, isomaltose, maltose, ribose, arabinose, xylose, starch, oligosaccharides and polysaccharides, such as starch, glycogen, cellulose, chitin; amine containing compounds other than 6-ACA, in particular amino acids other than 6-ACA, proteins and other peptides; organic acids; inorganic salts, in particular phosphate salts, sulphate salts; and biomass (cells).

Usually, the mixture containing 6-ACA is subjected to one or more pre-treatment steps prior to cyclising the 6-ACA. Usually, biomass is separated from the 6-ACA. Further, water and/or further components stemming from the fermentation medium may be separated from 6-ACA. The concentration at which 6-ACA is subjected to cyclisation (the cyclisation concentration) or at least the concentration of a feed comprising 6-ACA that is introduced into a cyclisation reactor (the feed concentration) may be chosen within wide limits.

Usually, the 6-ACA cyclisation or feed concentration is at least 50 g/l 6-ACA, in particular at least 100 g/l, more in particular at least 150 g/l or at least 250 g/l. Even more preferably, the 6-ACA cyclisation or feed concentration is at least 250 g/l, and most preferably it is at least 400 g/l. The upper limit is not particularly critical. It is in principle tolerable that the feed comprises solid 6-ACA, as long as the feed remains processable. Usually, the 6-ACA cyclisation or feed concentration is 950 g/l or less, in particular 750 g/l or less, more in particular 500 g/l or less.

When referred herein to a “6-ACA cyclisation or feed concentration” this includes 6-ACA monomers and 6-ACA oligomers, which oligomers may have formed if the feed is heated prior to cyclisation.

Although in principle essentially all residual components from the culture medium (nutrients, non-reacted raw materials and other components other than water and 6-ACA) may have been removed before cyclising 6-ACA, in practice cyclisation of 6-ACA usually takes place in the presence of one or more residual components other than water. Usually, the total concentration of residual components (other than water) will be less than 40 wt. %, in particular less than 30 wt. %, more in particular less than 20 wt. % or less than 10 wt. %, as a percentage of the 6-ACA cyclisation or feed concentration. The total concentration of residual components (other than water) in particular may be at least 2 wt. %, at least 5 wt. % or at least 8 wt. %, as a percentage of the 6-ACA cyclisation or feed concentration. The balance, if any, is formed by water.

In particular, based on experiments wherein 6-ACA in a fermentation medium was cyclised, the inventors contemplate that it is advantageous to carry out the cyclisation in the absence of carbohydrates, or at a low concentration of carbohydrates. Accordingly, in a preferred method, the mixture comprises less than 5 g/l of carbohydrates. In a specifically preferred embodiment the mixture containing 6-ACA comprises less than 2 g/l, in particular less than 1 g/l, more in particular less than 0.5 g/l of carbohydrates.

In an embodiment, a carbon source different from carbohydrates is used as a carbon source for the 6-ACA in the fermentative process, e.g. a fatty acid, amino acids, glycerol, acetic acid, ethanol. Of such carbon sources it is contemplated that they may be less prone to react with 6-ACA or caprolactam to form a side-product that may be difficult to remove.

In a further embodiment, a fed-batch type fermentation process is used. Herein, the carbon source, such as a carbohydrate or another carbon source, is gradually added to the fermentation medium, during the preparation of 6-ACA.

In order to obtain a mixture containing 6-ACA prepared by fermenting a carbohydrate, which product has a relatively low carbohydrate content, a separation step may be carried out to separate 6-ACA from the carbohydrate.

In accordance with the invention it is not necessary to carry out a fermentative process at a total carbohydrate to 6-aminocaproic acid of 0.03 or less, nor to carry out the whole fermentative process at a low carbohydrate concentration. It is sufficient that said ratio is 0.03 or less in the recovered mixture comprising the 6-ACA that is cyclised. It is advantageous though to at least end the fermentative process at a ratio of 0.03 or less and/or to end the fermentation at a low carbohydrate concentration, in particular a concentration of less than 5 g/l. By limiting the feed of carbohydrate (or not feeding any carbohydrate), at some point in the fermentative process, the microorganisms will cause the concentration of carbohydrate to be lower, as they metabolise the carbohydrate as a carbon source (e.g. to produce the 6-ACA). Thus said ratio and/or low carbohydrate concentration can be reached, also when starting from conditions wherein said ratio and/or carbohydrate concentration are higher.

In an embodiment, the fermentative process is carried out throughout the fermentative process or at least at the end of the fermentative process under carbon-limited conditions, i.e. under conditions wherein growth of the microorganism is limited by limiting the supply of the carbon nutrient. Such method is in particular considered advantageous, since a specific separation step to separate 6-ACA from excess nutrient, may be omitted, if desired. It is envisaged that carbon limited conditions are in particular favourable in case a carbohydrate is used as a carbon source. Carbon-limited conditions (wherein inter alia carbohydrate concentration is low) may directly result in a low carbohydrate concentration in the mixture containing 6-ACA. In a specific embodiment, the fermentative process is not carried out under non-carbon-limited conditions prior to carrying said process out under carbon-limited conditions. Thus, initially growing conditions may be employed (during which initially a carbon source may be fed to the system), which may be advantageous for the production rate of 6-ACA. The conditions then become carbon-limited when the micro-organism have converted so much carbon source that the concentration becomes a carbon limiting concentration (usually after stopping any carbon source feed).

In an embodiment, the recovery of the mixture containing 6-ACA comprises separating 6-ACA from cell mass in a pre-treatment step, in particular by a technique selected from the group of tangential flow filtration, microfiltration, other forms of filtration, and centrifugation.

In an embodiment, the recovery of the mixture containing 6-ACA comprises separating 6-ACA from one or more other amine containing compounds in a pre-treatment step, in particular from one or more compounds selected from the group of other amino acids, peptides and proteins.

It is contemplated that in particular in a method wherein the mixture containing 6-ACA has a low carbohydrate content, a separate step to separate one or more amine containing compounds and 6-ACA may be omitted, whilst maintaining a relatively high yield and/or allowing a relatively simple purification of the caprolactam product obtained by cyclisation.

In an embodiment, the recovery of the mixture containing 6-ACA comprises separating 6-ACA and one or more polymers, such as one or more polymers selected from the group of polysaccharides, polypeptides and proteins. Ultrafiltration is particularly suitable to that purpose, wherein 6-ACA is recovered in the filtrate. For the ultrafiltration a filter is typically chosen with a cut-off above the molecular weight of 6-ACA and below the molecular weight of the polymer(s) that are to be separated from the 6-ACA.

In an embodiment, the recovery of the mixture containing 6-ACA comprises a water removal step prior cyclising 6-ACA. In general, only part of the water will be removed and remaining water in the mixture containing 6-ACA can contribute to the steam in which presence cyclisation of 6-ACA takes place. Removal of water may in particular be accomplished by evaporation of water.

In an embodiment, the recovery comprises separating 6-ACA and one or more salts. However, a method according to the invention may be carried out without a step wherein 6-ACA is separated from one or more salts. It is contemplated that the cyclisation may suitably be carried out in the presence of a salt, e.g. a phosphate or a sulphate salt, and that in at least some embodiments, the presence thereof may be beneficial in that the salt may act as a cyclisation catalyst.

The cyclisation process may in principle be based on a known cyclisation process, e.g. as described in U.S. Pat. Nos. 6,194,572 or 3,658,810.

Usually, cyclisation is carried out at a temperature in the range of from 250 to 400° C. In particular, the temperature may be 275° C. or more, 280° C. or more, 290° C. or more, or 300° C. or more. In particular, the temperature may be 375° C. or less, 360° C. or less, 340° C. or less, or 330° C. or less. A relatively low temperature is preferred for a low occurrence of side-reactions; especially above 330-340° C. decarboxylation and/or deamination of (e.g.) 6-ACA may become an issue. A relatively high temperature is preferred for a fast reaction rate. In view of these considerations, the temperature may in particular be chosen in the range of 290-330° C.

Usually, cyclisation is carried out at a pressure in the range of from 0.3 to 2 MPa. In particular, the pressure may be 0.5 MPa or more, 0.8 MPa or more, or 1.0 MPa or more. In particular, the pressure may be 1.5 MPa or less, 1.4 MPa or less, or 1.2 MPa or less. A relatively high pressure is advantageous for a high reaction rate. The pressure may be increased by feeding pressurised steam in the cyclisation reactor, wherein 6-ACA is cyclised. A consequence thereof is that the higher the pressure, the more water condensate will generally be formed, diluting the product. In view of these considerations, the pressure may in particular be chosen in the range of from 0.8 to 1.5 MPa.

The invention further relates to a method for purifying caprolactam, comprising subjecting a product comprising caprolactam obtained in a method according to the invention to at least one distillation step, thereby obtaining a fraction enriched in caprolactam. Preferably such method comprises at least a distillation step to remove lights (i.e. compounds having a lower boiling point than caprolactam) and a distillation step to remove heavies (i.e. compounds having a higher boiling point than caprolactam), from caprolactam. Suitable process conditions may be based on methodology known in the art, e.g. from EP-A 1 062 203.

Preferably the fraction enriched in caprolactam, obtained by distillation, is subjected to a crystallisation step, thereby obtaining caprolactam crystals Caprolactam crystals may be isolated from the remaining liquid phase in a manner known per se, e.g. by filtration or centrifugation.

The isolated crystals may be further purified, e.g. by melting and flashing in a manner known per se.

The caprolactam may thereafter be used for preparing a polymer, in particular a polyamide, the preparation comprising polymerising caprolactam obtained in a method according to the invention, optionally in the presence of one or more further polymerisable compounds.

Regarding, the fermentative production of 6-ACA, it is observed that this can be done in a manner known per se.

In a specific embodiment, 6-ACA is fermentatively produced from 6-aminohex-2-enoic acid or 6-amino-2-hydroxy-hexanoic acid, e.g. using a host cell as described in WO 2005/068643 under fermentative conditions.

In a further specific embodiment, 6-ACA is produced from alpha-ketopimelic acid, using a biocatalyst having decarboxylase activity and/or aminotransferase activity, e.g. in a manner as disclosed in WO 2009/113855.

The invention will now be illustrated by means of a Comparative Example and some Examples, but is as such not restricted to the scope of the Examples.

### COMPARATIVE EXAMPLE A

Fermentation broth was obtained from a fermentation process with E. coli for production of a commercial enzyme. Biomass was removed from the broth by microfiltration. Bio-polymers, including the target product, then were removed by ultrafiltration. By adding 6-ACA to the remaining fermentation broth, a model fermentation broth for a 6-ACA fermentation process was prepared, wherein 6-ACA is obtained at a titer of 150 g/l. The total carbohydrate content in this mixture was 6.3 g/l (i.e. the carbohydrate to 6-ACA weight ratio was 0.042). The resulting product mixture was concentrated under vacuum in a forced circulation evaporator at 40° C. The concentrated mixture contained 48.3 wt. % water, 42.1 wt. % 6-ACA, 1.8 wt. % carbohydrates and 7.8 wt. % of other broth components (organic acids, inorganic ions, etc.).

1 Kilogram of the thus obtained concentrated product mixture was fed to a 2 liter stirred tank reactor. The reactor was closed and the contents were inertized by flushing with nitrogen. The reactor pressure controller in the vapour exit line in top of the reactor was kept at 1.2 MPa during the entire experiment. After starting the stirrer at 1000 r.p.m. the reactor contents were gradually heated up during approximately 25 minutes to about 315° C., using electric wall heating. During this period the water present in product mixture gradually evaporated and was condensed in a vapour cooler present in the vapour exit line. The recovered condensate fraction was weighed and analysed using HPLC for 6-ACA, CAP and the linear and cyclic oligomers thereof. When the reactor content reached the target temperature of about 315° C. a water feed was started and controlled at a rate of between 400 and 800 g/hr. This water was fed via a feed pipe beneath the stirrer where steam was formed in situ when the water came in contact with the hot reactor contents. Steam and steam-stripped products left the reactor via the vapour exit line at the top of the reactor. The condensed fractions were weighed and analysed by HPLC for content of CAP, 6-ACA and the linear and cyclic oligomers thereof. In this way it took approx. 5 hours to complete the reaction. The caprolactam yield obtained in this experiment was 67 mol % (calculated as the total of caprolactam analysed in the recovered product condensates relative to the overall amount of 6-ACA that was fed to the reactor originally).

### EXAMPLE 1

A fermentation broth was prepared in a similar way as described in comparative example A, with the only difference that the original fermentation was prolonged for sufficient time so as to obtain a lower residual carbohydrate content in the fermentation broth. In this way a similar model fermentation mixture was prepared as in comparative example A, but now the carbohydrate concentration of this model broth was 1.3 g/l and the carbohydrate to 6-ACA weight ratio was 0.0087. Using the same procedure for 6-ACA conversion to caprolactam as described in comparative example A, the caprolactam yield finally obtained was 85 mol %.

### EXAMPLE 2

Example 1 was repeated, with the difference that the residual carbohydrate concentration in the finally obtained model fermentation broth was even further reduced to 0.3 g/l (by prolonging the fermentation time); the carbohydrate to 6-ACA weight ratio was thereby reduced to 0.0020. Using the same procedure for 6-ACA conversion to caprolactam as described in comparative example A, the caprolactam yield finally obtained was 94 mol %.

Above examples show that high caprolactam yields are achievable if the carbohydrate to 6-ACA weight ratio in the fermentation broth is reduced to a low value.

1. Method for preparing caprolactam comprising recovering a mixture containing 6-aminocaproic acid from a culture medium comprising biomass, which culture medium comprises more than one carbohydrate, and thereafter cyclising the 6-aminocaproic acid in the presence of superheated steam, thereby forming caprolactam, wherein the weight to weight ratio of total carbohydrate to 6-aminocaproic acid in said mixture is 0.03 or less.

2. Method according to claim 1, wherein the mixture comprises less than 5 g/l.

3. Method according to claim 1, wherein the mixture comprises less than 2 g/l carbohydrates.

4. Method according to claim 1, wherein the mixture comprises less than 0.5 g/l carbohydrates.

5. Method according to claim 1, wherein the 6-aminocaproic acid is prepared microbiologically, which microbiological preparation is at least ended under carbon-limited conditions.

6. Method according to claim 1, wherein the 6-aminocaproic acid is prepared microbiologically, which microbiological preparation is at least ended at a total carbohydrate concentration in the culture medium of less than 5 g/l.

7. Method according to claim 1, wherein the 6-amino caproic is separated from biomass by at least one technique selected from the group of tangential flow filtration, microfiltration, other forms of filtration, and centrifugation.

8. Method according to claim 1, wherein the recovery of the mixture comprises separating 6-aminocaproic acid and one or more polymers.

9. Method according to claim 8, wherein the 6-aminocaproic acid is separated from one or more polymers by ultrafiltration.

10. Method according to claim 1, wherein the mixture is subjected to a water removal step prior cyclising 6-aminocaproic acid.

11. Method according to claim 1, wherein the cyclisation is carried out at a temperature in the range of from 250 to 400° C.

12. Method according to claim 1, wherein the cyclisation is carried out at a pressure in the range of from 0.3 to 2 MPa.

13. Method for purifying caprolactam, comprising performing the method according to claim 1 and subjecting the caprolactam to at least one distillation step, thereby obtaining a fraction enriched in caprolactam.

14. Method according to claim 13, wherein one or more compounds having a lower boiling point than caprolactam and one or more compounds having a higher boiling point than caprolactam are separated from caprolactam by distillation, thereby obtaining a fraction enriched in caprolactam, and subjecting said fraction to a crystallisation step, thereby obtaining caprolactam crystals.

15. Method for preparing a polymer, comprising performing the method of claim 1 and polymerising the caprolactam.

16. Method according to claim 1, wherein the 6-aminocaproic acid is prepared microbiologically, which microbiological preparation is at least ended at a total carbohydrate concentration in the culture medium of less than 2 g/l.

17. Method according to claim 1, wherein the 6-aminocaproic acid is prepared microbiologically, which microbiological preparation is at least ended at a total carbohydrate concentration in the culture medium of less than 0.5 g/l.

18. Method according to claim 8, wherein said one or more polymers is selected from the group of polysaccharides, polypeptides and proteins.

19. Method according to claim 15, wherein the polymerizing is in the presence of one or more further polymerisable compounds.
